(19) United States
(12) Patent Application Publication
(10) Pub. No.: US 2025/0235495 A1
(43) Pub. Date: Jul. 24, 2025

(54) DUAL TREATMENT METHOD OF PRODUCING GINSENG CONCENTRATE WITH REDUCED BITTERNESS USING COMPOSITE ADSORBENT AND MIXED CYCLODEXTRIN

(71) Applicant: DAEDONG KOREA GINSENG CO., LTD, Chungcheongnam-do (KR)

(72) Inventors: SUNG SOO JANG, Chungcheongnam-do (KR); KUN HEE LEE, Chungcheongnam-do (KR); SUNG-KEUN CHOI, Chungcheongnam-do (KR); CHANG-SOON LEE, Chungcheongbuk-do (KR); BYEONG-SEON JEON, Daejeon (KR); JAE HYUNG PARK, Daejeon (KR); HYE JEONG JEON, Daejeon (KR)

(21) Appl. No.: 18/929,857

(22) Filed: Oct. 29, 2024

(30) Foreign Application Priority Data
Jan. 22, 2024 (KR) 10-2024-0009280

(51) Int. Cl. A61K 36/258; A61K 47/40; B01D 11/02; B01D 15/08; B01J 20/12; B01J 20/14; B01J 20/20; B01J 20/28

(52) U.S. Cl. CPC A61K 36/258; A61K 47/40; B01D 11/0284; B01D 11/0288; B01D 15/08; B01J 20/12; B01J 20/14; B01J 20/20; B01J 20/28052; A61K 2236/15; A61K 2236/331; A61K 2236/39; A61K 2236/51; A61K 2236/55

(57) ABSTRACT

A method of producing ginseng concentrate includes passing a ginseng extract through a composite adsorbent to produce a ginseng extract treated with the composite adsorbent, adding cyclodextrin mixture powder to the prepared ginseng extract treated with the composite adsorbent followed by stirring, allowing to stand, and filtering, and concentrating the filtered ginseng extract, and also a ginseng concentrate produced by the method.

### CROSS-REFERENCE TO RELATED APPLICATION AND CLAIM OF PRIORITY

This application claims the benefit under 35 USC § 119 of Korean Patent Application No. 10-2024-0009280, filed on Jan. 22, 2024, in the Korean Intellectual Property Office, the entire disclosure of which is incorporated herein by reference for all purposes.

### BACKGROUND

### 1. Technical Field

The present invention relates to a method of producing ginseng concentrate including: passing a ginseng extract through a composite adsorbent mixed with acid clay, activated carbon, and diatomaceous earth to produce a ginseng extract treated with the composite adsorbent; preparing a cyclodextrin mixture powder by mixing α-cyclodextrin, β-cyclodextrin, and γ-cyclodextrin; adding the prepared cyclodextrin mixture powder to the prepared ginseng extract treated with the composite adsorbent followed by stirring, allowing to stand, and filtering; and concentrating the filtered ginseng extract, and also a ginseng concentrate produced by the method.

### 2. Background Art

Korean ginseng (Panax ginseng) has long been regarded as a superior medicinal herb in traditional oriental medicine. Recently, its effectiveness and medicinal benefits have been scientifically validated through pharmacological and clinical studies, which has led to its increasing popularity as a natural functional food, beyond the traditional concept of a tonic to strengthen basic physical health. From a traditional oriental medicine perspective, the benefits of ginseng include replenishing vital energy, preventing fatigue, enhancing blood production, and restoring pulse strength. Additionally, according to the records, it is believed to nourish the mind, calm the spirit, generate bodily fluids, alleviate thirst, strengthen the lungs, ease asthma, fortify the digestive system, stop diarrhea, expel toxins, and heal sores. Modern pharmacological research has demonstrated that ginseng has an effect of boosting the body's non-specific resistance to stress and playing a role in maintaining homeostasis of a human body. Other known benefits include improving hypertension, enhancing insulin activity, reducing blood sugar levels in diabetic mice, promoting RNA synthesis, protein synthesis, carbohydrate and lipid metabolism in rat liver, and providing anti-cancer effects.

Ginseng consumption is primarily concentrated in regions like China, Japan, Korea, and Southeast Asia, and, by age group, it is most prevalent among middle-aged and older adults. The limited consumer base for ginseng is attributed to its bitter taste and distinctive earthy aroma, often described as a “soil smell,” which is known to be particularly unappealing to Western consumers. Therefore, there is a growing need to develop ginseng health products having reduced bitterness and earthy aroma, making them more appealing to a broader audience, including people of all ages and backgrounds globally. However, masking ginseng's characteristic bitterness is challenging, and there is a problem that the process of reducing bitterness often results in a decrease in the functional component, ginsenosides, which are essential for its health benefits.

Types of adsorbents include activated carbon, activated clay, acid clay, zeolite, diatomaceous earth, and bentonite. Activated carbon is one of the most commonly used adsorbents, and it is primarily employed for solvent recovery and removing organic impurities. Acid clay is a clay with acidic properties, similar in appearance to bentonite but with a higher silica content and stronger adsorption capacity. Furthermore, diatomaceous earth, formed from diatom deposits, is used as an adsorbent, filtration agent, abrasive, and in the purification of pharmaceuticals and food products.

Cyclodextrins are used as pharmaceutical and food additives with purposes such as stabilizing unstable components, masking undesirable tastes and odors of a component with unpleasant odor or the like, reducing side effects through the use of inclusion compounds based on inclusion reaction, prolonging the pharmaceutical effects, solubilizing poorly soluble substances, and aiding in dissolution. Cyclodextrins are a type of oligosaccharide made up of multiple glucopyranose repeating units, and they maintain a circular, three-dimensional molecular structure. Depending on the number of glucose residues, they are named alpha-cyclodextrin (6 glucose units), beta-cyclodextrin (7 units), or gamma-cyclodextrin (8 units), each with different cavity sizes. In addition, the unique donut-shaped structure of cyclodextrins makes the interior surface of their cavity hydrophobic and the exterior surface hydrophilic in aqueous solutions, allowing them to encapsulate various organic and inorganic compounds within the cavity.

Korean Patent Application Publication No. 2012-0110667 discloses a method of producing ginseng beverage with reduced characteristic flavor and bitterness of ginseng, and Korean Patent Registration No. 1194265 discloses a method for removing bitter taste from ginseng or red ginseng concentrate using deep sea water. However, those methods differ from the method of producing ginseng concentrate of the present invention, which is based on the dual treatment using composite adsorbent and mixed cyclodextrin.

### SUMMARY

The present invention is devised in response to the aforementioned needs. Object of the invention is to provide, to have a ginseng concentrate that maintains the excellent pharmacological effects of ginseng while reducing its bitterness, improving palatability for easier consumption, and ensuring superior formulation stability, a method of producing ginseng concentrate that retains or enhances its functional components while reducing bitterness by optimizing the manufacturing conditions including the preparation of a ginseng extract, the preparation of a treatment solution, the adsorbent treatment, and the cyclodextrin treatment.

To solve the problems that are described in the above, the present invention provides a method of producing ginseng concentrate including: (1) adding water to ginseng and carrying out extraction to prepare a treatment solution; (2) immersing ginseng in the treatment solution prepared in the step (1) above, and then taking it out and drying it; (3) preparing a ginseng extract by adding water to ground ginseng obtained by grinding the dried ginseng of the step (2) above and carrying out extraction; (4) preparing a composite adsorbent by mixing acid clay, activated carbon, and diatomaceous earth; (5) passing the ginseng extract prepared in the step (3) through the composite adsorbent prepared in the step (4) to produce a ginseng extract treated with the composite adsorbent; (6) mixing α-cyclodextrin, β-cyclodextrin, and γ-cyclodextrin to prepare cyclodextrin mixture powder; (7) adding the cyclodextrin mixture powder prepared in the step (6) to the ginseng extract treated with the composite adsorbent, which is prepared in the step (5), followed by stirring, allowing to stand, and filtering; and (8) concentrating the filtered ginseng extract of the step (7).

The present invention further provides a ginseng concentrate produced by the aforementioned method.

Ginseng concentrate produced by the method of the present invention, i.e., dual treatment using composite adsorbent and mixed cyclodextrin, is easy to ingest by having reduced characteristic bitter taste of ginseng, and has improved sensory properties, making it a ginseng concentrate more preferred by consumers. Furthermore, during the processing stages, the key functional component of ginseng, ginsenosides, are not diminished; in fact, content of some ginsenosides is even enhanced, allowing for the production of a high-quality ginseng concentrate.

### BRIEF DESCRIPTION OF THE DRAWINGS

FIG. 1 is a photographic image of the composite adsorbent column of the present invention.

FIG. 2 shows the production process of ginseng concentrates (white ginseng, red ginseng, black ginseng) according to the present invention.

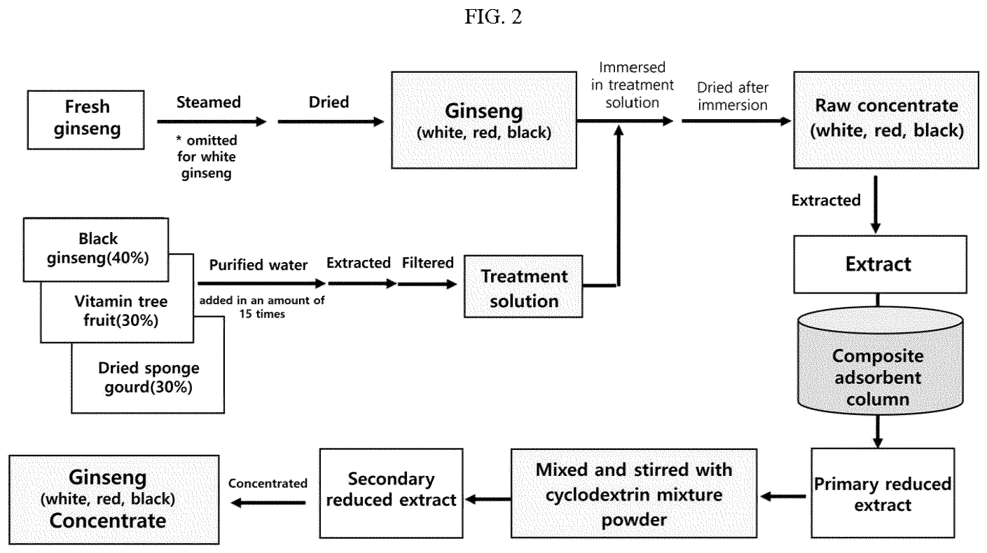

FIG. 3 shows the results of principal component analysis (PCA) for comparing the taste patterns of red ginseng concentrates from Preparation example 1 (RG-A), Comparative example 1 (RG-C), and Comparative example 2 (RG-B).

FIG. 4 shows the results of discriminant factorial analysis (DFA) for comparing the taste patterns of red ginseng concentrates from Preparation example 1 (RG-A), Comparative example 1 (RG-C), and Comparative example 2 (RG-B).

FIG. 5 shows the quantified results of the sensitivity of each electronic tongue sensor in analyzing the red ginseng concentrates from Preparation example 1 (RG-A), Comparative example 1 (RG-C), and Comparative example 2 (RG-B).

### DETAILED DESCRIPTION

To achieve the object described in the above, the present invention provides a method of producing ginseng concentrate including:
(1) adding water to ginseng and carrying out extraction to prepare a treatment solution;
(2) immersing ginseng in the treatment solution prepared in the step (1) above, and then taking it out and drying it;
(3) preparing a ginseng extract by adding water to ground ginseng obtained by grinding the dried ginseng of the step (2) above and carrying out extraction;
(4) preparing a composite adsorbent by mixing acid clay, activated carbon, and diatomaceous earth;
(5) passing the ginseng extract prepared in the step (3) through the composite adsorbent prepared in the step (4) to produce a ginseng extract treated with the composite adsorbent;
(6) mixing α-cyclodextrin, β-cyclodextrin, and γ-cyclodextrin to prepare cyclodextrin mixture powder;
(7) adding the cyclodextrin mixture powder prepared in the step (6) to the ginseng extract treated with the composite adsorbent, which is prepared in the step (5), followed by stirring, allowing to stand, and filtering; and
(8) concentrating the filtered ginseng extract of the step (7).

With regard to the method of producing ginseng concentrate according to the present invention, the treatment solution in the step (1) can preferably be prepared by adding water to ginseng at a ratio of 10 to 20 times (v/w) and carrying out extraction at a temperature of 80° C. to 90° C. for 1 to 3 hours. More preferably, the production can be made by performing the extraction with water added at a ratio of 15 times (v/w) to ginseng and carrying out extraction at 85° C. for 2 hours.

The ginseng used for preparing the treatment solution can preferably be one or more selected from the group consisting of fresh ginseng, red ginseng, dried ginseng, white ginseng, black ginseng, Taeguk ginseng, dried and twisted ginseng, wood-cultivated ginseng, cultivated ginseng, wild ginseng cultivated roots, sprouted ginseng, and wild ginseng, with black ginseng being more preferable. Specifically, the black ginseng can be prepared by steaming ginseng at a temperature of 90° C. to 110° C. for 1 to 3 hours, repeating the process 3 to 4 times, and then drying it at a temperature of 45° C. to 55° C. until the moisture content is within 10 to 20% (v/w). More specifically, it can be prepared by steaming ginseng at 100° C. for 2 hours, repeating the process 3 times, and then drying it at 50° C. until the moisture content is within 15% (v/w).

Additionally, during the preparation of the treatment solution, a vitamin tree and sponge gourd can be also used. More specifically, a mixture of black ginseng, vitamin tree, and sponge gourd can be prepared at a weight ratio of 3.5-4.5:2.5-3.5:2.5-3.5, to which water is added at a ratio of 10 to 20 times (v/w) and extraction is carried out at 80° C. to 90° C. for 1 to 3 hours to produce the treatment solution. Even more specifically, a mixture of black ginseng, vitamin tree, and sponge gourd at a weight ratio of 4:3:3 can have water added at a ratio of 15 times (v/w) and be extracted at 85° C. for 2 hours to produce the treatment solution. Pre-treating the ginseng with the treatment solution prepared under the aforementioned conditions can effectively reduce the characteristic bitterness of ginseng while enhancing palatability due to the taste and aroma of the treatment solution.

Furthermore, in the method of producing ginseng concentrate of the present invention, the step (2) preferably involves immersing ginseng in the treatment solution at 25° C. to 35° C. for 25 to 35 minutes, and then taking it out and drying it at 45° C. to 55° C. until the moisture content is 10 to 20% (v/w). More preferably, ginseng can be immersed in the treatment solution at 30° C. for 30 minutes, then removed and dried at 50° C. until the moisture content is 15% (v/w). Immersing ginseng in the treatment solution, and then drying it under those conditions can improve the smooth taste and swallowing of ginseng.

The ginseng used in the step (2) is preferably selected from the group consisting of fresh ginseng, red ginseng, dried ginseng, white ginseng, black ginseng, Taeguk ginseng, dried and twisted ginseng, wood-cultivated ginseng, cultivated ginseng, wild ginseng cultivated roots, sprouted ginseng, and wild ginseng, and more preferably, it may be red ginseng or black ginseng. The red ginseng can be specifically prepared by steaming the ginseng at 90° C. to 110° C. for 1 to 3 hours, and then drying it at 45° C. to 55° C. until the moisture content is 10 to 20% (v/w). More specifically, the red ginseng can be prepared by steaming the ginseng at 100° C. for 2 hours, and then drying it at 50° C. until the moisture content is within 15% (v/w).

Moreover, in the method of producing ginseng concentrate according to the present invention, the ginseng extract in the step (3) is preferably prepared by adding water at a ratio of 5 to 10 times (v/w) to ground ginseng that has been dried before, and then carrying out extraction at 85° C. to 90° C. for 1 to 3 hours. More preferably, the ginseng extract can be prepared by adding water at a ratio of 8 times (v/w) to the ground ginseng that has been dried before, and then carrying out extraction at 85° C. to 90° C. for 2 hours.

Moreover, in the method of producing ginseng concentrate according to the present invention, the composite adsorbent in the step (4) is preferably prepared by mixing acid clay, activated carbon, and diatomaceous earth at a weight ratio of 1.8-2.2:0.8-1.2:0.8-1.2. More preferably, the composite adsorbent can be prepared by mixing acid clay, activated carbon, and diatomaceous earth at a weight ratio of 2:1:1.

Moreover, in the method of producing ginseng concentrate according to the present invention, when the ginseng extract is passed through the composite adsorbent to produce a ginseng extract treated with the composite adsorbent as described in the step (5), the ginseng extract can be passed through the composite adsorbent at a flow rate of 12 to 16 L/hr (bed volume) and a space velocity of 9 to 13 cm³/hr, and more specifically, the ginseng extract can be passed through the composite adsorbent at a flow rate of 14 L/hr (bed volume) and a space velocity of 11 cm³/hr. Treating the extract with the adsorbent under those conditions effectively reduces the off-flavors and bitterness of the ginseng extract while preventing the decrease in ginsenoside content. However, if the adsorption treatment conditions are outside the specified range, there is a risk that the effective components of ginseng will be adsorbed on to the adsorbent, leading to a decrease in ginsenoside content or minimal effects on bitterness reduction.

Moreover, in the method of producing ginseng concentrate according to the present invention, the cyclodextrin mixture powder in the step (6) can preferably be produced by mixing α-cyclodextrin, β-cyclodextrin, and γ-cyclodextrin at a weight ratio of 1.5-2.5:4.5-5.5:2.5-3.5. More preferably, α-cyclodextrin, β-cyclodextrin, and γ-cyclodextrin can be mixed at a weight ratio of 2:5:3. Using α-cyclodextrin, β-cyclodextrin, and γ-cyclodextrin in this specific ratio effectively reduces the bitterness of ginseng while further enhancing its palatability.

Moreover, in the method of producing ginseng concentrate according to the present invention, the step (7) preferably involves adding cyclodextrin mixture powder at a concentration of 3 to 7% (w/v) to the ginseng extract treated with the composite adsorbent, followed by stirring and allowing to stand at 4° C. to 7° C. for 10 to 14 hours, and then filtering. More preferably, cyclodextrin mixture powder can be added at 5% (w/v) to the ginseng extract treated with the composite adsorbent, stirred, and allowed to stand at 4° C. to 7° C. for 12 hours before filtering. Treating with cyclodextrin in this manner effectively reduces harmful components and bitterness in the ginseng concentrate without decreasing the ginsenoside content in the concentrate.

Moreover, in the method of producing ginseng concentrate according to the present invention, in the step (8), the concentration is preferably carried out by concentrating the ginseng extract at 50° C. to 60° C. to a Brix level of 60 to 70. More preferably, the ginseng extract can be concentrated at 55° C. to a Brix level of 65.

More specifically, the method of producing ginseng concentrate according to the present invention may include the following steps of:
(1) adding water at a ratio of 10 to 20 times (v/w) to ginseng and carrying out extraction at 80° C. to 90° C. for 1 to 3 hours to prepare a treatment solution;
(2) immersing ginseng in the treatment solution prepared in the step (1) above at 25° C. to 35° C. for 25 to 35 minutes, and then taking it out and drying it at 45° C. to 55° C. until the moisture content is 10 to 20% (v/w);
(3) preparing a ginseng extract by adding water at a ratio of 5 to 10 times (v/w) to ground ginseng obtained by grinding the dried ginseng of the step (2) above, and then carrying out extraction at 85° C. to 90° C. for 1 to 3 hours;
(4) preparing a composite adsorbent by mixing acid clay, activated carbon, and diatomaceous earth at a weight ratio of 1.8-2.2:0.8-1.2:0.8-1.2;
(5) passing the ginseng extract prepared in the step (3) through the composite adsorbent prepared in the step (4) to produce a ginseng extract treated with the composite adsorbent;
(6) mixing α-cyclodextrin, β-cyclodextrin, and γ-cyclodextrin at a weight ratio of 1.5-2.5:4.5-5.5:2.5-3.5 to prepare cyclodextrin mixture powder;
(7) adding the cyclodextrin mixture powder prepared in the step (6) in an amount of 3 to 7% (w/v) to the ginseng extract treated with the composite adsorbent, which is prepared in the step (5), followed by stirring, allowing to stand at 4° C. to 7° C. for 10 to 14 hours, and then filtering; and
(8) concentrating the filtered ginseng extract of the step (7) at 50° C. to 60° C. to a Brix level of 60 to 70.

Even more specifically, the method may include the following steps of:
(1) adding water at a ratio of 15 times (v/w) to ginseng and carrying out extraction at 85° C. for 2 hours to prepare a treatment solution;
(2) immersing ginseng in the treatment solution prepared in the step (1) above at 30° C. for 30 minutes, and then taking it out and drying it at 50° C. until the moisture content is 15% (v/w);
(3) preparing a ginseng extract by adding water at a ratio of 8 times (v/w) to ground ginseng obtained by grinding the dried ginseng of the step (2) above, and then carrying out extraction at 85° C. to 90° C. for 2 hours;
(4) preparing a composite adsorbent by mixing acid clay, activated carbon, and diatomaceous earth at a weight ratio of 2:1:1;
(5) passing the ginseng extract prepared in the step (3) through the composite adsorbent prepared in the step (4) to produce a ginseng extract treated with the composite adsorbent;
(6) mixing α-cyclodextrin, β-cyclodextrin, and γ-cyclodextrin at a weight ratio of 2:5:3 to prepare cyclodextrin mixture powder;
(7) adding the cyclodextrin mixture powder prepared in the step (6) in an amount of 5% (w/v) to the ginseng extract treated with the composite adsorbent, which is prepared in the step (5), followed by stirring, allowing to stand at 4° C. to 7° C. for 12 hours, and then filtering; and
(8) concentrating the filtered ginseng extract of the step (7) at 55° C. to a Brix level of 65.

The present invention further provides a ginseng concentrate produced by the aforementioned method.

Hereinbelow, the present invention is explained in greater detail in view of Examples and Production examples. However, the following Examples and Production examples are given only for exemplification of the present invention and it is evident that the scope of the present invention is not limited by them.

### EXAMPLES

### Preparation Example 1. Red Ginseng Concentrate (RG-A)

(1) Red ginseng was prepared by steaming fresh ginseng once at 100° C. for 2 hours, and then drying it at 50° C. until the moisture content became not more than 15% (v/w).
(2) Ginseng was steamed three times at 100° C. for 2 hours, then dried at 50° C. to moisture content of not more than 15% (v/w) to produce black ginseng. Subsequently, purified water was added at 15 times (v/w) the dried black ginseng, which was then extracted at 85° C. for 2 hours and filtered to prepare a treatment solution.
(3) The red ginseng prepared in the step (1) was immersed in the treatment solution prepared in the step (2) at 30° C. for 30 minutes, then taken out and dried at 50° C. until the moisture content became not more than 15% (v/w).
(4) The dried red ginseng from the step (3) was ground to yield ground red ginseng, to which purified water was added at 8 times (v/w). The mixture was then extracted at 85° C. to 90° C. for 2 hours, followed by filtration to produce a red ginseng extract.
(5) A composite adsorbent column was prepared by mixing acid clay, activated carbon, and diatomaceous earth at a weight ratio of 2:1:1.
(6) The red ginseng extract prepared in the step (4) was passed through the composite adsorbent column prepared in the step (5) at a bed volume (BV) of 14 L/hr and a space velocity (SV) of 11 cm³/hr to produce a composite adsorbent-treated red ginseng extract. As described herein, the bed volume (BV) refers to the amount of liquid passing through the column per hour (L/hr), and the space velocity (SV) refers to the flow rate, calculated by dividing the amount of liquid passing through (cm³/hr) by the column volume (cm³).
(7) α-Cyclodextrin, β-cyclodextrin, and γ-cyclodextrin were mixed at a weight ratio of 2:5:3 to prepare a cyclodextrin mixture powder.
(8) The cyclodextrin mixture powder prepared in the step (7) was added at 5% (w/v) to the composite adsorbent-treated red ginseng extract prepared in the step (6), stirred, and left to stand at 4° C. to 7° C. for 12 hours, followed by filtration.
(9) The filtered red ginseng extract from the step (8) was concentrated at 55° C. to a Brix level of 65 to produce a red ginseng concentrate.

### Preparation Example 2. Black Ginseng Concentrate

(1) Black ginseng was prepared by steaming fresh ginseng three times at 100° C. for 2 hours, and then drying it at 50° C. until the moisture content became not more than 15% (v/w).
(2) Ginseng was steamed three times at 100° C. for 2 hours, then dried at 50° C. to moisture content of not more than 15% (v/w) to produce black ginseng. Subsequently, purified water was added at 15 times (v/w) the dried black ginseng, which was then extracted at 85° C. for 2 hours and filtered to prepare a treatment solution.
(3) The black ginseng prepared in the step (1) and the treatment solution prepared in the step (2) were used to produce a black ginseng concentrate by following the same procedure as the steps (3) to (9) of Preparation example 1.

### Preparation Example 3. Red Ginseng Concentrate

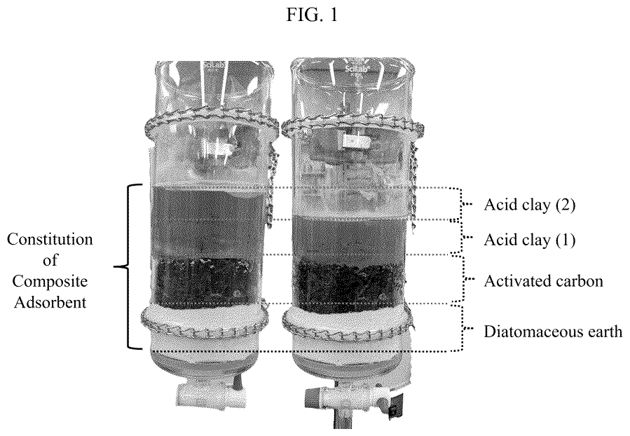

(1) Red ginseng was prepared by steaming fresh ginseng once at 100° C. for 2 hours, and then drying it at 50° C. until the moisture content became not more than 15% (v/w).
(2) Ginseng was steamed three times at 100° C. for 2 hours, then dried at 50° C. to moisture content of not more than 15% (v/w) to produce black ginseng. The dried black ginseng was then mixed with vitamin tree fruit and dried sponge gourd at a weight ratio of 4:3:3. Subsequently, purified water was added at 15 times (v/w) the mixture, which was then extracted at 85° C. for 2 hours and filtered to prepare a treatment solution.
(3) The red ginseng prepared in the step (1) and the treatment solution prepared in the step (2) were used to produce a red ginseng concentrate by following the same procedure as the steps (3) to (9) of Preparation example 1 (FIG. 1).

### Preparation Example 4. Black Ginseng Concentrate

(1) Black ginseng was prepared by steaming fresh ginseng three times at 100° C. for 2 hours, and then drying it at 50° C. until the moisture content became not more than 15% (v/w).
(2) Ginseng was steamed three times at 100° C. for 2 hours, then dried at 50° C. to moisture content of not more than 15% (v/w) to produce black ginseng. The dried black ginseng was then mixed with vitamin tree fruit and dried sponge gourd at a weight ratio of 4:3:3. Subsequently, purified water was added at 15 times (v/w) the mixture, which was then extracted at 85° C. for 2 hours and filtered to prepare a treatment solution.
(3) The black ginseng prepared in the step (1) and the treatment solution prepared in the step (2) were used to produce a black ginseng concentrate by following the same procedure as the steps (3) to (9) of Preparation example 1 (FIG. 1).

### Comparative Example 1. Red Ginseng Concentrate (RG-C)

(1) Red ginseng was prepared by steaming fresh ginseng once at 100° C. for 2 hours, and then drying it at 50° C. until the moisture content became not more than 15% (v/w).
(2) Ginseng was steamed three times at 100° C. for 2 hours, then dried at 50° C. to moisture content of not more than 15% (v/w) to produce black ginseng. Subsequently, purified water was added at 15 times (v/w) the dried black ginseng, which was then extracted at 85° C. for 2 hours and filtered to prepare a treatment solution.
(3) The red ginseng prepared in the step (1) was immersed in the treatment solution prepared in the step (2) at 30° C. for 30 minutes, then taken out and dried at 50° C. until the moisture content became not more than 15% (v/w).
(4) The dried red ginseng from the step (3) was ground to yield ground red ginseng, to which purified water was added at 8 times (v/w). The mixture was then extracted at 85° C. to 90° C. for 2 hours, followed by filtration to produce a red ginseng extract.
(5) The red ginseng extract prepared in the step (4) was concentrated at 55° C. to a Brix level of 65 to produce a red ginseng concentrate.

### Comparative Example 2. Red Ginseng Concentrate (RG-B)

(1) Red ginseng was prepared by steaming fresh ginseng once at 100° C. for 2 hours, and then drying it at 50° C. until the moisture content became not more than 15% (v/w).
(2) Ginseng was steamed three times at 100° C. for 2 hours, then dried at 50° C. to moisture content of not more than 15% (v/w) to produce black ginseng. Subsequently, purified water was added at 15 times (v/w) the dried black ginseng, which was then extracted at 85° C. for 2 hours and filtered to prepare a treatment solution.
(3) The red ginseng prepared in the step (1) was immersed in the treatment solution prepared in the step (2) at 30° C. for 30 minutes, then taken out and dried at 50° C. until the moisture content became not more than 15% (v/w).
(4) The dried red ginseng from the step (3) was ground to yield ground red ginseng, to which purified water was added at 8 times (v/w). The mixture was then extracted at 85° C. to 90° C. for 2 hours, followed by filtration to produce a red ginseng extract.
(5) A composite adsorbent column was prepared by mixing acid clay, activated carbon, and diatomaceous earth at a weight ratio of 2:1:1.
(6) The red ginseng extract prepared in the step (4) was passed through the composite adsorbent column prepared in the step (5) at a bed volume (BV) of 14 L/hr and a space velocity (SV) of 11 cm³/hr to produce a composite adsorbent-treated red ginseng extract.
(7) The red ginseng extract prepared in the step (6) was concentrated at 55° C. to a Brix level of 65 to produce a red ginseng concentrate.

### Comparative Example 3. Red Ginseng Concentrate

(1) Red ginseng was prepared by steaming fresh ginseng once at 100° C. for 2 hours, and then drying it at 50° C. until the moisture content became not more than 15% (v/w).
(2) Ginseng was steamed three times at 100° C. for 2 hours, then dried at 50° C. to moisture content of not more than 15% (v/w) to produce black ginseng. Subsequently, purified water was added at 15 times (v/w) the dried black ginseng, which was then extracted at 85° C. for 2 hours and filtered to prepare a treatment solution.
(3) The red ginseng prepared in the step (1) was immersed in the treatment solution prepared in the step (2) at 30° C. for 30 minutes, then taken out and dried at 50° C. until the moisture content became not more than 15% (v/w).
(4) The dried red ginseng from the step (3) was ground to yield ground red ginseng, to which purified water was added at 8 times (v/w). The mixture was then extracted at 85° C. to 90° C. for 2 hours, followed by filtration to produce a red ginseng extract.
(5) α-Cyclodextrin, β-cyclodextrin, and γ-cyclodextrin were mixed at a weight ratio of 2:5:3 to prepare a cyclodextrin mixture powder.
(6) The cyclodextrin mixture powder prepared in the step (5) was added at 5% (w/v) to the red ginseng extract prepared in the step (4), stirred, and left to stand at 4 to 7° C. for 12 hours, followed by filtration.
(7) The filtered red ginseng extract from the step (6) was concentrated at 55° C. to a Brix level of 65 to produce a red ginseng concentrate.

### Materials and Methods

### 1) Preparation of Composite Adsorbent Column

The composite adsorbent column was prepared using acid clay, activated carbon, and diatomaceous earth (FIG. 1). Acid clay is primarily composed of hydrous aluminum silicate obtained by refining montmorillonite clay minerals. When 1 g of acid clay is suspended in 500 mL of water, pH of the filtered liquid should be between 3.0 and 5.0, with a preferred acidity of 4.5. The moisture content should be not more than 15%, and preferably not more than 10%. Activated carbon is produced by carbonizing plant fibers such as sawdust, wood chips, or palm bark, followed by activation. Preferably, activated carbon should be sourced from palm bark. When 0.5 g of activated carbon is placed in a test tube and heated directly while blowing air through the mouth of test tube, it should combust without flames, generating combustion gases that produce a turbidity when passed through a potassium hydroxide solution. Additionally, when 0.1 g of activated carbon powder is mixed well with 10 mL of dilute methylene blue solution and two drops of dilute hydrochloric acid, and then filtered through quantitative filter paper, the resulting liquid should be colorless. Diatomaceous earth (diatomite) is a material composed of diatoms or siliceous shells of unicellular plants.

TABLE 1
Characteristics of activated carbon

| | | Apparent | Water | Thermal | Specific |
|---|---|---|---|---|---|
| | True density | density | permeability | conductivity | heat |
| Requirements | 2.00 to 2.30 | 0.1 to 0.8 | 0.0957 ml/cm² | 0.00013 to | 0.25 |
| Preferred | 2.05 to 2.15 | 0.2 to 0.3 | for 1 cm-thick | 0.00018 | |
| requirements | | | layer | gr · cal/cm²/sec/° C. | |
| | | | | at 100 to 900° C. | |

### 2) Determination of Taste Pattern Using E-Tongue Analysis

The red ginseng concentrate without any bitterness control (RG-C), the adsorbent-treated red ginseng concentrate (RG-B), and the adsorbent-treated and cyclodextrin-treated red ginseng concentrate (RG-A) were each diluted 100 times with distilled water. Using an electronic tongue (diluted solution 100 ml), test samples were measured for 120 seconds each, repeating the measurement five times.

The electronic tongue consists of seven sensors: AHS (sourness), CTS (saltiness), NMS (umami), PKS (sweetness), ANS (bitterness), CPS, and SCS, with CPS and SCS used as standards. Each of the seven sensors does not measure specific chemical components; rather, they detect the overall taste of the red ginseng concentrates for analysis. The sensor sensitivity was converted into taste scores ranging from 0 to 10. The taste component pattern analysis for each test sample was performed using discriminant function analysis (DFA) with the AlphaSoft 17 software provided by Alpha MOS (Toulouse, France).

### 3. Measurement of Ginsenoside Content

Content of ginsenosides was analyzed using HPLC (High Performance Liquid Chromatography) equipped with a UVD (Ultra Visible Detector), the analysis conditions are as shown in Table 2 below, and the formula for content calculation is as follows.
Ginsenoside content (mg/g)=S×(a×b)/Sample collection amount (g)
S: Concentration of individual ginsenoside in test solution (mg/ml)
a: Total amount of test solution (ml)
b: Dilution factor

TABLE 2
Conditions for ginsenoside analysis

| Device | GILSON system | |
|---|---|---|
| Detector | UV detector (203 nm) | |
| Column | Frontosil 120-5-C18-ace- | |
| | EPS | |
| | (4.6 mm × 250 mm, 5.0 μm) | |
| Column | 40° | C. |
| temperature | | |
| Mobile phase | Acetonitrile: D.W. | |
| | (gradient) | |
| Flow rate | 1.2 | ml/min |
| Injection amount | 20 | μl |

### 4. Sensory Evaluation

In a study involving 20 adult participants, each person was asked to consume each of the treated ginseng concentrates and evaluate their palatability. For the palatability evaluation, participants rated the intensity of sweetness, sourness, bitterness, saltiness, and spiciness, with higher scores given for stronger intensities. Additionally, overall preference was assessed, with higher scores awarded for better taste evaluations.

### Example 1. Results of Electronic Tongue Analysis

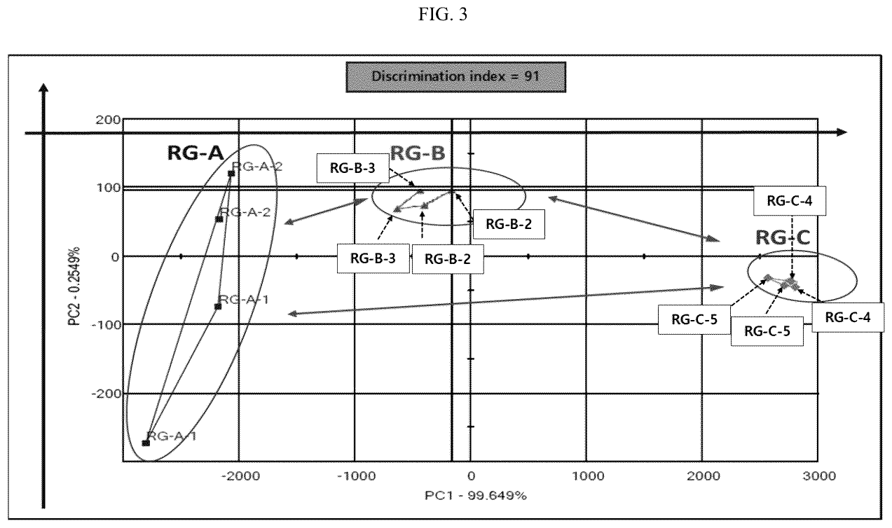

The results of the principal component analysis (PCA) comparing the taste patterns of the ginseng concentrates from Preparation example 1 (RG-A), Comparative example 1 (RG-C), and Comparative example 2 (RG-B) are shown in FIG. 3. The PCA results indicated that the ginseng concentrates RG-C, RG-B, and RG-A exhibited distinct differences in flavor profiles, with RG-A and RG-C being quite apart from each other in the analysis, while RG-A and RG-B, as well as RG-B and RG-C, displayed relatively closer distances.

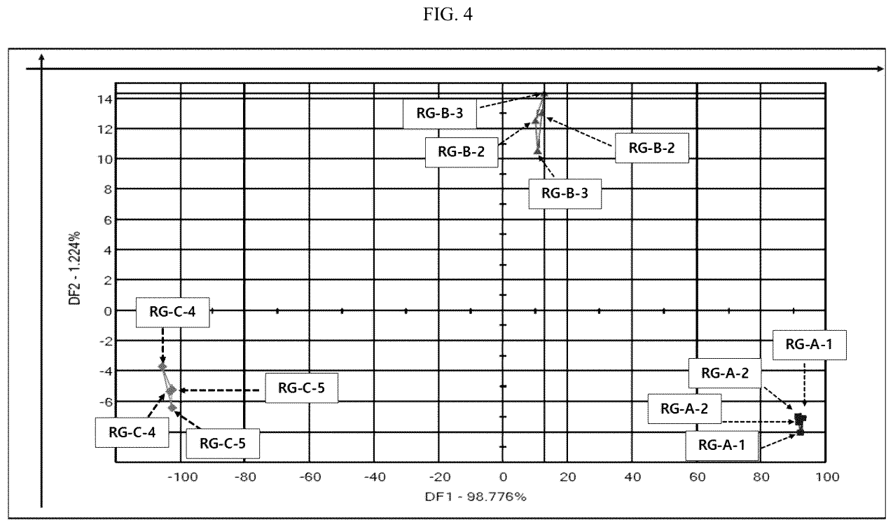

Additionally, the results of the discriminant factorial analysis (DFA), illustrated in FIG. 4, also showed the differences among RG-C, RG-A, and RG-B (FIG. 4). The distance values for each ginseng concentrate are presented in Table 3 shown below; a greater distance implies significant taste differences, while a shorter distance indicates greater similarity in taste profiles.

TABLE 3
Comparison of relative distance among different
test samples based on PCA and DFA analysis

| Product | Reference | Distances |
|---|---|---|
| RG-A | RG-B | 1906.06 |
| RG-A | RG-C | 5015.05 |
| RG-B | RG-C | 3119.84 |

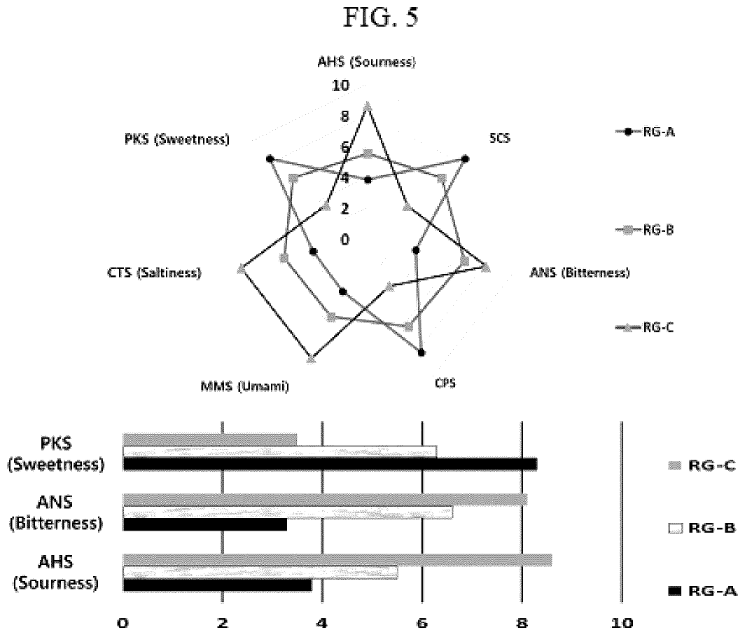

Additionally, the electronic tongue analysis results of the three test samples, which represent the relative comparison among the sample sensitivities for each sensor, indicated that RG-C exhibited relatively higher sensitivity levels for bitterness, sourness, sweetness, saltiness, and umami compared to both RG-B and RG-A. When comparing RG-B and RG-A, the ginseng concentrate that underwent dual treatment with adsorbent and cyclodextrin (RG-A) showed better taste profiles, particularly in terms of reduced bitterness and enhanced sweetness, compared to the concentrate which has been treated only with the adsorbent (RG-B) (FIG. 5).

### Example 2. Results of Ginsenoside Analysis

The ginsenoside analysis results for the red ginseng concentrates from Preparation example 1 and the Comparative examples are presented in Table 4 shown below. The results indicate that the ginseng concentrate from Preparation example 1 contains a higher total content of five ginsenosides compared to the concentrates from the Comparative examples.

TABLE 4
Results of ginsenoside analysis for red ginseng concentrates (mg/g)

| | | | | | | Total |
|---|---|---|---|---|---|---|
| | | | | | | of five |
| | | | | | | com- |
| | Re | Rb1 | Rg2 | Rb2 | Rd | ponents |
| Preparation example 1 | 5.73 | 12.99 | 1.11 | 5.99 | 2.93 | 28.75 |
| (adsorbent + cyclodextrin) | | | | | | |
| Comparative example 1 | 4.22 | 8.98 | 0.97 | 3.97 | 2.13 | 20.27 |
| (no treatment) | | | | | | |
| Comparative example 2 | 5.30 | 12.06 | 1.10 | 5.47 | 2.79 | 26.72 |
| (treated with adsorbent) | | | | | | |
| Comparative example 3 | 4.14 | 8.77 | 0.71 | 3.48 | 1.93 | 19.03 |
| (treated with cyclodextrin) | | | | | | |

### Example 3. Selection of Proper Type of Composite Adsorbent

The results of the sensory evaluation comparing the red ginseng concentrates, which have been obtained by treating a red ginseng extract with various types of composite adsorbent columns followed by concentration, specifically without cyclodextrin treatment, are presented in Table 5. The results indicate that the red ginseng concentrate treated with the composite adsorbent column composed of acid clay, activated carbon, and diatomaceous earth mixed at a weight ratio of 2:1:1 exhibited the lowest bitterness and the highest overall preference among the test samples evaluated.

TABLE 5
Sensory evaluation of red ginseng concentrates prepared with
various types of composite adsorbent

| | Sweet- | Sour- | Bitter- | Salti- | Spici- | Overall |
|---|---|---|---|---|---|---|
| Type | ness | ness | ness | ness | ness | preference |
| Not treated | 2.5 | 2.0 | 3.5 | 1.0 | 0.0 | 2.5 |
| with adsorbent | | | | | | |
| Acid clay: | 3.0 | 2.5 | 3.0 | 1.0 | 0.0 | 3.0 |
| activated | | | | | | |
| carbon: | | | | | | |
| diatomaceous | | | | | | |
| earth (1:1:1) | | | | | | |
| Acid clay: | 3.0 | 3.0 | 2.5 | 1.0 | 0.0 | 3.5 |
| activated | | | | | | |
| carbon: | | | | | | |
| diatomaceous | | | | | | |
| earth (2:1:1) | | | | | | |

### Example 4. Selection of Proper Type of Cyclodextrin

The sensory evaluation results comparing red ginseng concentrates obtained from red ginseng extracts which have been treated with various types of cyclodextrin (α, β, γ) followed by concentration (without adsorbent treatment) indicate that the red ginseng concentrate treated with a cyclodextrin mixture powder in 2:5:3 ratio of α-cyclodextrin, β-cyclodextrin, and γ-cyclodextrin exhibited the highest sweetness and the lowest bitterness, leading to the highest overall preference.

TABLE 6
Sensory evaluation of red ginseng concentrates prepared with
various types of cyclodextrin

| | Sweet- | Sour- | Bitter- | Salti- | Spici- | Overall |
|---|---|---|---|---|---|---|
| Type | ness | ness | ness | ness | ness | preference |
| Without any | 2.5 | 2.0 | 3.5 | 1.0 | 0.0 | 2.5 |
| treatment | | | | | | |
| β:γ (5:5) | 3.0 | 2.5 | 2.5 | 1.0 | 0.0 | 3.0 |
| α:β:γ (2:5:3) | 3.0 | 3.0 | 2.0 | 1.0 | 0.0 | 3.5 |
| α:β:γ (3:5:2) | 3.0 | 2.5 | 2.5 | 1.0 | 0.0 | 3.0 |
| α:β (5:5) | 2.5 | 2.5 | 2.5 | 1.0 | 0.0 | 3.0 |

### Example 5. Comparison Between Single Treatment and Combined Treatment

Through Examples 3 and 4, the optimal ratios of adsorbent and cyclodextrin were determined, and a sensory evaluation was conducted on the red ginseng extract subjected to the combined treatment with adsorbent and cyclodextrin. The results indicated that the combined treatment of adsorbent and cyclodextrin significantly enhanced the sweetness of the red ginseng concentrate compared to treatments using adsorbent only or cyclodextrin only. Additionally, the combined treatment effectively reduced sourness and bitterness, leading to an overall improvement in the palatability of the red ginseng concentrate.

TABLE 7
Sensory evaluation of red ginseng concentrates prepared by various
types of treatment (combined treatment vs. single treatment)

| | Sweet- | Sour- | Bitter- | Salti- | Spici- | Overall |
|---|---|---|---|---|---|---|
| Type | ness | ness | ness | ness | ness | preference |
| Preparation example 1 | 3.5 | 2.5 | 1.5 | 1.0 | 0.0 | 4.0 |
| (adsorbent + | | | | | | |
| cyclodextrin) | | | | | | |
| Comparative example 2 | 3.0 | 3.0 | 2.5 | 1.0 | 0.0 | 3.5 |
| (adsorbent) | | | | | | |
| Comparative example 3 | 3.0 | 3.0 | 2.0 | 1.0 | 0.0 | 3.5 |
| (cyclodextrin) | | | | | | |

### Example 6. Comparison Among Various Types of Materials for Treatment Solution

The results of the sensory evaluation conducted with the red ginseng concentrates from Preparation example 1 and Preparation example 3 are summarized in Table 8 shown below. The results indicate that Preparation example 3, which utilized black ginseng, vitamin tree, and sponge gourd all together in the treatment solution, effectively reduced 5 both bitterness and sourness compared to the red ginseng concentration of Preparation example 1. Furthermore, Preparation example 3 received a higher preference score, indicating its enhanced palatability.

TABLE 8
Sensory evaluation of red ginseng concentrates prepared using various
types of treatment solution

| | Sweet- | Sour- | Bitter- | Salti- | Spici- | Overall |
|---|---|---|---|---|---|---|
| Type | ness | ness | ness | ness | ness | preference |
| Preparation | 3.5 | 2.5 | 1.5 | 1.0 | 0.0 | 4.0 |
| example 1 | | | | | | |
| (black ginseng | | | | | | |
| treatment | | | | | | |
| solution) | | | | | | |
| Preparation | 3.5 | 2.0 | 1.0 | 1.0 | 0.0 | 4.5 |
| example 3 | | | | | | |
| (black ginseng + | | | | | | |
| vitamin | | | | | | |
| tree + sponge gourd | | | | | | |
| treatment solution) | | | | | | |

What is claimed is:

1. A method of producing ginseng concentrate, the method comprising:
(1) adding water to ginseng and carrying out extraction to prepare a treatment solution;
(2) immersing ginseng in the treatment solution prepared in the step (1) above, and then taking it out and drying it;
(3) preparing a ginseng extract by adding water to ground ginseng obtained by grinding the dried ginseng of the step (2) above and carrying out extraction;
(4) preparing a composite adsorbent by mixing acid clay, activated carbon, and diatomaceous earth;
(5) passing the ginseng extract prepared in the step (3) through the composite adsorbent prepared in the step (4) to produce a ginseng extract treated with the composite adsorbent;
(6) mixing α-cyclodextrin, β-cyclodextrin, and γ-cyclodextrin to prepare cyclodextrin mixture powder;
(7) adding the cyclodextrin mixture powder prepared in the step (6) to the ginseng extract treated with the composite adsorbent, which is prepared in the step (5), followed by stirring, allowing to stand, and filtering; and
(8) concentrating the filtered ginseng extract of the step (7).

2. The method of claim 1, wherein the ginseng is one or more selected from the group consisting of fresh ginseng, red ginseng, dried ginseng, white ginseng, black ginseng, Taeguk ginseng, dried and twisted ginseng, wood-cultivated ginseng, cultivated ginseng, wild ginseng cultivated roots, sprouted ginseng, and wild ginseng.

3. The method of claim 1, wherein the step (4) comprising preparing the composite adsorbent by mixing the acid clay, the activated carbon, and the diatomaceous earth at a weight ratio of 1.8-2.2:0.8-1.2:0.8-1.2; and
the step (6) comprises mixing the α-cyclodextrin, the β-cyclodextrin, and the γ-cyclodextrin at a weight ratio of 1.5-2.5:4.5-5.5:2.5-3.5 to prepare the cyclodextrin mixture powder.

4. The method of claim 3, wherein the treatment solution of the step (1) is prepared by carrying out extraction after adding water to a mixture of ginseng, vitamin tree, and sponge gourd.

5. The method of claim 3, wherein the step (1) comprises adding the water at a ratio of 10 to 20 times (v/w) to the ginseng and carrying out the extraction at 80° C. to 90° C. for 1 to 3 hours to prepare the treatment solution;
the step (2) comprises immersing the ginseng in the treatment solution prepared in the step (1) above at 25° C. to 35° C. for 25 to 35 minutes, and then taking it out and drying it at 45° C. to 55° C. until the moisture content is 10 to 20% (v/w);
the step (3) comprises preparing the ginseng extract by adding the water at a ratio of 5 to 10 times (v/w) to the ground ginseng obtained by grinding the dried ginseng of the step (2) above, and then carrying out the extraction at 85° C. to 90° C. for 1 to 3 hours;
the step (7) comprises adding the cyclodextrin mixture powder prepared in the step (6) in an amount of 3 to 7% (w/v) to the ginseng extract treated with the composite adsorbent, which is prepared in the step (5), followed by stirring, allowing to stand at 4° C. to 7° C. for 10 to 14 hours, and then filtering; and
the step (8) comprises concentrating the filtered ginseng extract of the step (7) at 50° C. to 60° C. to a Brix level of 60 to 70.

6. The method of claim 4, wherein the step (1) comprises adding the water at a ratio of 10 to 20 times (v/w) to a mixture of ginseng, vitamin tree, and sponge gourd mixed at a weight ratio of 3.5-4.5:2.5-3.5:2.5-3.5 and carrying out the extraction at 80° C. to 90° C. for 1 to 3 hours to prepare the treatment solution;
the step (2) comprises immersing the ginseng in the treatment solution prepared in the step (1) above at 25° C. to 35° C. for 25 to 35 minutes, and then taking it out and drying it at 45° C. to 55° C. until the moisture content is 10 to 20% (v/w);
the step (3) comprises preparing a ginseng extract by adding water at a ratio of 5 to 10 times (v/w) to the ground ginseng obtained by grinding the dried ginseng of the step (2) above, and then carrying out the extraction at 85° C. to 90° C. for 1 to 3 hours;
the step (4) comprises preparing the composite adsorbent by the mixing acid clay, the activated carbon, and the diatomaceous earth at a weight ratio of 1.8-2.2:0.8-1.2:0.8-1.2;
the step (6) comprises mixing the α-cyclodextrin, the β-cyclodextrin, and the γ-cyclodextrin at a weight ratio of 1.5-2.5:4.5-5.5:2.5-3.5 to prepare the cyclodextrin mixture powder;
the step (7) comprising adding the cyclodextrin mixture powder prepared in the step (6) in an amount of 3 to 7% (w/v) to the ginseng extract treated with the composite adsorbent, which is prepared in the step (5), followed by stirring, allowing to stand at 4° C. to 7° C. for 10 to 14 hours, and then filtering; and
the step (8) comprises concentrating the filtered ginseng extract of the step (7) at 50° C. to 60° C. to a Brix level of 60 to 70.

7. A ginseng concentrate produced by the method of claim 1.

8. A method of producing ginseng concentrate, the method comprising:
(1) adding water at a ratio of 10 to 20 times (v/w) to a mixture of ginseng, vitamin tree, and sponge gourd mixed at a weight ratio of 3.5-4.5:2.5-3.5:2.5-3.5 and carrying out extraction at 80° C. to 90° C. for 1 to 3 hours to prepare a treatment solution;
(2) immersing ginseng in the treatment solution prepared in the step (1) above at 25° C. to 35° C. for 25 to 35 minutes, and then taking it out and drying it at 45° C. to 55° C. until the moisture content is 10 to 20% (v/w);
(3) preparing a ginseng extract by adding water at a ratio of 5 to 10 times (v/w) to ground ginseng obtained by grinding the dried ginseng of the step (2) above, and then carrying out extraction at 85° C. to 90° C. for 1 to 3 hours;
(4) preparing a composite adsorbent by mixing acid clay, activated carbon, and diatomaceous earth at a weight ratio of 1.8-2.2:0.8-1.2:0.8-1.2;
(5) passing the ginseng extract prepared in the step (3) through the composite adsorbent prepared in the step (4) to produce a ginseng extract treated with the composite adsorbent;
(6) mixing α-cyclodextrin, β-cyclodextrin, and γ-cyclodextrin at a weight ratio of 1.5-2.5:4.5-5.5:2.5-3.5 to prepare cyclodextrin mixture powder;
(7) adding the cyclodextrin mixture powder prepared in the step (6) in an amount of 3 to 7% (w/v) to the ginseng extract treated with the composite adsorbent, which is prepared in the step (5), followed by stirring, allowing to stand at 4° C. to 7° C. for 10 to 14 hours, and then filtering; and
(8) concentrating the filtered ginseng extract of the step (7) at 50° C. to 60° C. to a Brix level of 60 to 70.
